(19) United States
(12) Patent Application Publication
(10) Pub. No.: US 2021/0038121 A1
(43) Pub. Date: Feb. 11, 2021

(54) SYSTEM AND METHOD TO MONITOR AN EXERCISE POSTURE OF AT LEAST ONE USER

(71) Applicant: WELLNESYS INC., Suwanee, GA (US)

(72) Inventors: Muralidhar SOMISETTY, Bangalore (IN); Pranav KANUPARTHI, Bangalore (IN); Sankar DAS IGA, Bangalore (IN)

(21) Appl. No.: 16/609,224

(22) PCT Filed: Nov. 2, 2018
(86) PCT No.: PCT/IB2018/058620

§ 371 Date: Oct. 29, 2019

(30) Foreign Application Priority Data
Feb. 22, 2018 (IN) 201841006784

(51) Int. Cl. A61B 5/11; A61B 5/107; A61B 5/00; A63B 21/00

(52) U.S. Cl. CPC A61B 5/1116; A63B 21/4037; A61B 5/6892; A61B 5/1071

(57) ABSTRACT

System and method to monitor an exercise posture of at least one user are provided. The system includes a flexible substrate placed on top of a mat coupled with one or more contact sensors configured to generate a contact signal upon making a contact of at least one part of a body of the corresponding at least one user with the mat while performing the at least one exercise on the mat. The system further includes a processing subsystem configured to receive the contact signal, and to receive an angle signal generated by at least one inertial sensor upon measuring an angle of at least one user while performing the at least one exercise, to generate a combined signal, to compare the combined signal with a pre-defined set of exercise posture signals and to monitor the exercise posture of the at least one user.

This International Application claims priority from a complete patent application filed in India having Patent Application No. 201841006784, filed on Feb. 22, 2018 and titled “SYSTEM AND METHOD TO MONITOR AN EXERCISE POSTURE OF AT LEAST ONE USER”.

### BACKGROUND

Embodiments of the present disclosure relate to monitoring exercise posture, and more particularly to a system and method to monitor an exercise posture of at least one user.

Physical exercise is any kind of bodily activity which enhances or maintains physical fitness and overall wellness of a body. Further, modern technologies provide an interactive platform for enhancing the physical exercise of a user with the help of multimedia.

In one approach, a system is implemented on an exercise mat to guide a user's performance of exercises on the exercise mat. The system provides graphical illustrations on the exercise mat for the user to perform the exercise based on the graphical illustrations provided by the system on the exercise mat. However, in such system the exercise done by the user is not monitored and hence the user is not guided. Also, the graphical illustration may end up confusing the user to perform the exercise. Further, such system may also intrude the privacy of the user. Also, in such system, the existing mat has to be discarded in order to make use of such a system.

In another approach, making use of the modern technology a system to detect the posture of the user is built, wherein the system uses a plurality of pressure sensors which is fabricated on a substrate layer which is further imposed on the exercise mat. The system detects the amount of pressure applied by the user on the exercise mat and compares the applied pressure with a pre-defined pressure gradient. However, in such system the contact of the user with the exercise mat is not detected accurately and hence fails to detect if the user is performing the exercise in an exact way in which the exercise has to be performed or not. Further the system uses a single layer of substrate to fabricate the plurality of sensors which may lead to high power consumption and high leakage current in the substrate layer. Further, the system does not detect and measure the angle or position of the body of the user while performing the exercise when the user is not in contact with the exercise mat.

Hence, there is a need for an improved system and method to monitor an exercise posture of at least one user.

### BRIEF DESCRIPTION

In accordance with one embodiment of the disclosure, a system to monitor an exercise posture of at least one user is provided. The system includes a mat configured to allow the at least one user to perform at least one exercise. The system also includes a flexible substrate placed on top of the mat, wherein the flexible substrate includes one or more contact sensors connected to each other in a form of a matrix, and the one or more sensors are configured to generate a contact signal upon making a contact of at least one part of a body of the corresponding at least one user with the mat while performing the at least one exercise on the mat. The system further includes a processing subsystem operatively coupled to the flexible substrate. The processing subsystem is configured to receive the contact signal generated by the one or more contact sensors. The processing subsystem is also configured to receive an angle signal generated by at least one inertial sensor upon measuring an angle of the at least one part of the body of the corresponding at least one user when the at least one user makes the contact with the mat while performing the at least one exercise on the mat. The system further includes a storage device operatively coupled to the flexible substrate and the processing subsystem. The storage device is configured to store a pre-defined set of exercise posture signals. The storage device is also configured to receive and store the contact signal generated upon making a contact of the at least one part of the body of the corresponding at least one user with the mat. The storage device is further configured to receive and store the angle signal generated upon measuring the angle of the at least one part of the body of the corresponding at least one user when the at least one user makes the contact with the mat. The processing subsystem is also configured to combine the contact signal and the angle signal to generate a combined signal. The processing subsystem is further configured to compare the combined signal with the pre-defined set of exercise posture signals. The processing subsystem is further configured to monitor the exercise posture of the at least one user.

In accordance with another embodiment of the disclosure, a method for monitoring an exercise posture of the at least one user is provided. The method includes storing a pre-defined set of exercise posture signals. The method also includes generating a contact signal upon making a contact of at least one part of the body of the corresponding at least one user with the mat while performing at least one exercise. The method further includes receiving an angle signal generated upon measuring an angle of the at least one part of the body of the corresponding at least one user when the at least one user makes the contact with the mat. The method further includes combining the contact signal and the angle signal and generating a combined signal. The method further comparing the combined signal with the pre-defined set of exercise posture signals. The method further includes monitoring the exercise posture of the at least one user.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a block diagram of a system to monitor an exercise posture of at least one user in accordance with an embodiment of the present disclosure;

FIG. 2 is an exemplary embodiment representing a system to monitor an exercise posture of at least one user of FIG. 1 in accordance with an embodiment of the present disclosure;

FIG. 3 is another exemplary embodiment representing a system to monitor an exercise posture of at least one user of FIG. 1 in accordance with an embodiment of the present disclosure; and

FIG. 4 is a process flow for monitoring an exercise posture of the at least one user in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or sub-systems or elements or structures or components preceded by “comprises . . . a” does not, without more constraints, preclude the existence of other devices, sub-systems, elements, structures, components, additional devices, additional sub-systems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present disclosure relate to system and method to monitor an exercise posture of at least one user is provided. The system includes a mat configured to allow the at least one user to perform at least one exercise. The system also includes a flexible substrate placed on top of the mat, wherein the flexible substrate includes one or more contact sensors connected to each other in a form of a matrix, and the one or more sensors are configured to generate a contact signal upon making a contact of at least one part of a body of the corresponding at least one user with the mat while performing the at least one exercise on the mat. The system further includes a processing subsystem operatively coupled to the flexible substrate. The processing subsystem is configured to receive the contact signal generated by the one or more contact sensors. The processing subsystem is also configured to receive an angle signal generated by at least one inertial sensor upon measuring an angle of the at least one part of the body of the corresponding at least one user when the at least one user makes the contact with the mat while performing the at least one exercise on the mat. The system further includes a storage device operatively coupled to the flexible substrate and the processing subsystem. The storage device is configured to store a pre-defined set of exercise posture signals. The storage device is also configured to receive and store the contact signal generated upon making a contact of the at least one part of the body of the corresponding at least one user with the mat. The storage device is further configured to receive and store the angle signal generated upon measuring the angle of the at least one part of the body of the corresponding at least one user when the at least one user makes the contact with the mat. The processing subsystem is also configured to combine the contact signal and the angle signal to generate a combined signal. The processing subsystem is further configured to compare the combined signal with the pre-defined set of exercise posture signals. The processing subsystem is further configured to monitor the exercise posture of the at least one user.

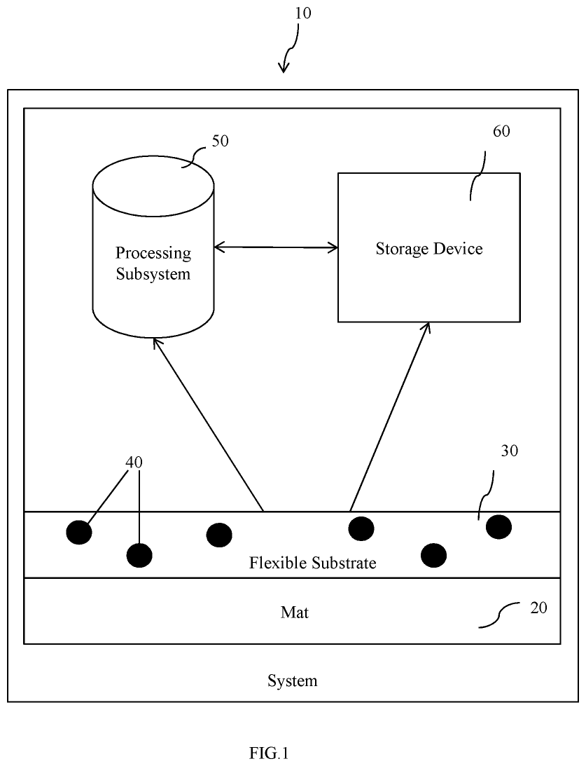

FIG. 1 is a block diagram of a system to monitor an exercise posture of at least one user in accordance with an embodiment of the present disclosure. The system (10) includes a mat (20) configured to allow the at least one user to perform at least one exercise. In one embodiment, the mat (20) may be a Pilate mat, a yoga mat, a fitness mat or a wellness mat. In another embodiment, the at least one exercise may be yoga, a gym, a gymnastic or a pliates. In yet another embodiment, the at least one user may be a person who may be learning to perform the at least one exercise. In yet another embodiment, the at least one user may be the person who may perform the at least one exercise regularly.

The system (10) also includes a flexible substrate (30) placed on top of the mat (20), wherein the flexible substrate (30) includes one or more contact sensors (40) connected to each other in a form of a matrix. The one or more contact sensors (40) are configured to generate a contact signal upon making a contact of at least one part of a body of the corresponding at least one user with the mat (20) while performing the at least one exercise on the mat (20). As used herein, the contact sensors are a type of sensors which responds to a contact of an object or a user on a sensor contact face. Further, contact sensors detect the object or the user and measure dimensions based on a detected position. In one embodiment, the one or more contact sensors may use at least one transducer for a sensing operation. In such embodiment, the one or more contact sensors (40) may be used to detect a change in a position, a velocity, an acceleration, a force or a torque of the at least one user.

In one embodiment, the flexible substrate (30) may be fabricated using an expanded polyethylene (EPE) sheet. In such embodiment, the flexible substrate (30) may include a first layer. Further, the first layer may be fabricated with at least one conducting path, wherein the conducting path may be configured to withstand an electric power within the conducting path.

Further, the flexible layer may also include a second layer, wherein the second layer may be fabricated with one or more contact sensors (40). In such embodiment, the one or more contact sensors (40) may be connected to each other in a form of a matrix. Further, the flexible substrate (30) may include a third layer which may be fabricated in-between the first layer and the second layer. The third layer may be configured to insulate the conducting path of the first layer from a matrix connection of the second layer. In such embodiment, the third layer may be an insulating layer.

In one embodiment, the third layer may include a plurality of slots. Further, the plurality of slots may be configured to detect the one or more contact sensors (40) on the second layer and to make a connectivity of the one or more contact sensors (40) with the conducting path of the first layer. Further, with reference to the above motioned embodiment, the second layer may include at least one supporting structure, wherein the at least one supporting structure may include a power supply, wherein the power supply may be configured to supply the electric power to the conducting path of the first layer. In one specific embodiment, the at least one supporting structure may also include a processing device operatively coupled to the one or more contact sensors (40). In such embodiment, the processing device may be configured to process the contact signal which may be generated by the one or more contact sensors (40).

In one specific embodiment, the flexible substrate (30) may be fabricated in a form of a hollow circular cover. In such embodiment, the mat (20) may be inserted into the hollow circular cover in such a way that a part of the flexible substrate (30) which may be fabricated with the one or more contact sensors (40) may be placed on top of the mat (20) on which the at least one user may perform the at least one exercise. In one embodiment, the at least one user may be asked to perform the at least one exercise to calibrate a height and a weight of the at least one user before an actual at least one exercise may be performed by the at least one user. In one specific embodiment, the flexible substrate (30) may act as an intelligent cover for an existing mat (20), on which the at least one exercise may be performed.

The system (10) further includes a processing subsystem (50) operatively coupled to the flexible substrate (30). The processing subsystem (50) is configured to receive the contact signal generated by the one or more contact sensors (40). In one embodiment, the at least one user may make the contact with the mat (20) through the at least one part of the body of the at least one user to perform the at least one exercise. Further, based on the contact made by the at least one part of the body, the one or more contact sensors (40) may generate a contact signal. Further, a generated contact signal may be transmitted to the processing subsystem (50).

In another embodiment, the contact signal which may be generated by the one or more contact sensors (40) may be transmitted to the processing device of the flexible substrate for processing of the generated contact signal. Further, a processed contact signal may be transmitted to the processing subsystem (50) for further monitoring of the contact signal. In such embodiment the contact signal may be transmitted to the processing subsystem (50) through a wireless transmission device. In one embodiment, the wireless transmission device may be a Bluetooth device, a Bluetooth low energy (BLE) device or a wireless fidelity (Wi-Fi) device. In one embodiment, the contact signal generated may be an analog signal. In such embodiment, an analog to digital converter may be operatively coupled to the one or more sensors (40) which may be configured to convert an analog contact signal into a digital contact signal.

The processing subsystem (50) is also configured to receive an angle signal generated by at least one inertial sensor upon measuring an angle of the at least one part of the body of the corresponding at least one user when the at least one user makes the contact with the mat (20) while performing the at least one exercise on the mat (20). In one embodiment, the at least one inertial sensor may be at least one accelerometer sensor, at least one gyroscopic sensor or at least one magnetic sensor. As used herein inertial sensors are sensors whose sensing parameters are based on inertia of the object. Further, accelerometer sensors are electromechanical sensors which are used to measure a plurality of acceleration forces of the object.

Also, the gyroscopic sensors also known as angular velocity sensors are a type of sensors used to sense an angular velocity of the object, wherein the angular velocity is a change in a rotational angle of the object per unit time. Further, the magnetic sensor is a type of sensor used to measure magnetism of a ferromagnetic material or a direction, strength or a relative change of a magnetic field of the object at a particular location.

Further, with reference to the above mentioned embodiment, the angle of the at least one part of the body of the user may be sensed by the at least one inertial sensor while the at least one user may perform the at least one exercise. In another embodiment, a posture of the at least one part of the body of the at least one user may be sensed by the at least one inertial sensor while the at least one user may perform the at least one exercise. Further, a sensed angle and a sensed posture of the at least one part of the body may be combined together to generate an angle signal. Further, a generated angle signal from the at least one inertial sensor may be transmitted to the processing subsystem (50) for further processing and measuring of the angle signal. In such embodiment, the angle signal may be transmitted to the processing subsystem (50) through the wireless transmission device. In one embodiment, the wireless transmission device may be the Bluetooth device, the Bluetooth low energy (BLE) device or the wireless fidelity (Wi-Fi) device.

The system (10) further includes a storage device (60) operatively coupled to the flexible substrate (30) and the processing subsystem (50). The storage device is configured to store a pre-defined set of exercise posture signals. In one embodiment, the pre-defined set of exercise posture signals may be stored in a remote server such as a cloud server. In such embodiment, at least one user expert may perform at least one exercise on the mat (20) and may measure a set of exercise posture signals and may store a measured set of exercise posture signals in the storage device (60) as the pre-defined set of exercise posture signals.

In one specific embodiment, the system (10) may further detect the angle or the posture of the at least one part of the body of the at least one user automatically. Further, based on an automatically detected angle or posture of the at least one part of the body of the at least one user, the system (10) may classify the at least one exercise performed by the at least one user on the mat depending on the pre-defined set of exercise posture signals. In such embodiment, the pre-defined set of exercise posture signals may also include a type of the at least one exercise.

In one embodiment, the at least one user expert may be an expert in performing the at least one exercise. In another embodiment, the at least one user expert may be a trainer of the at least one exercise to the at least one user. In one embodiment, the pre-defined set of exercise posture signals measured by the at least one user expert may be used as a reference signal to monitor the exercise posture of the at least one user.

The storage device (60) is also configured to receive and store the contact signal generated upon making a contact of the at least one part of the body of the corresponding at least one user with the mat (20). In one embodiment, the contact signal which may be generated by the one or more contact sensors (40) may be transmitted to the processing subsystem (50) for the processing of the contact signal. Further, the processed contact signal may be stored in the storage device (60) for further references. In another embodiment, the contact signal which may be generated by the one or more contact sensors (40) may be first transmitted to the storage device (60). Further, the contact signal which may be stored in the storage device (60) may be transmitted to the processing subsystem (50) for further measuring an analysis of the contact signal.

The storage device (60) is further configured to receive and store the angle signal generated upon measuring an angle of the at least one part of the body of the corresponding at least one user when the at least one user makes the contact with the mat (20). In one embodiment, the angle signal which may be generated by the at least one inertial sensor may be transmitter to the processing subsystem (50) for the processing of the angle signal. Further, a processed angle signal may be stored in the storage device (60) for further references. In another embodiment, the angle signal which may be generated by the at least one angle sensor may be first transmitted to the storage device (60). Further, the angle signal which may be stored in the storage device (60) may be transmitted to the processing subsystem (50) for further measuring an analysis of the angle signal.

Further, the processing subsystem (50) is configured to combine the contact signal and the angle signal to generate a combined signal. In one embodiment, the contact signal which may be generated by the one or more contact sensors (40) may be combined with the angle signal which may be generated by the at least one inertial sensor to and may generate a combined signal. In such embodiment, the combined signal may be generated to measure an accurate angle and an accurate position of the at least one part of the body of the user while performing the at least one exercise on the mat (20).

In one specific embodiment, the processing subsystem (50) may combine the contact signal and the angle signal using a kalman filter method or a complementary filter method. As used herein, the kalman filter which is also known as linear quadratic estimation (LQE) which measures a series of measurement of parameters in a defined time interval to produce an accurate measurement of the parameter. Further, the complementary filter is a type of steady state kalman filter. In one embodiment, the combining of the contact signal and the angle signal may result in a quaternion angle representation of the combined signal. In such embodiment, a resulted combined signal may be converted into a 3-dimensional (3D) angle representation which may be in terms of a roll, a pitch and a yaw. As used herein, quaternion is a type of number system that extends a complex numbers and can be applied to mechanics in three-dimensional space. Further, the roll, the pitch and the yaw are the three dimensions in an axes with dimensions

The processing subsystem (50) is also configured to compare the combined signal with the pre-defined set of exercise posture signals. In one embodiment, the combined signal which may be generated by the processing subsystem (50) may be compared with the pre-defined set of exercise posture signals to determine if the sensed contact signal and the sensed angle signal are accurate to the pre-defined set of exercise posture signals. Further, in such embodiment, a comparison of the combined signal with the pre-defined set of exercise posture signals may determine if the at least one exercise performed by the at least one user is performed in an appropriate way in which the at least one exercise may be performed or not. In one embodiment, the processing subsystem (50) may compare the combined signal with the pre-defined set of exercise posture signals using a machine learning model. In such embodiment, the machine learning model may be artificial intelligence. As used herein, machine learning is a type of study which gives a computer system an ability learn and perform things without being explicitly programmed. In one embodiment, the machine learning model may use a deep neural network method or an artificial neural network method which may be trained using the pre-defined set of exercise posture signals.

The processing subsystem (50) is further configured to monitor the exercise posture of the at least one user. In one embodiment, the at least one exercise performed by the at least one user may be monitored to check if the at least one exercise performed by the at least one user is performed rightly or not based on a comparison of the combined signal and the pre-defined set of exercise posture signals. In another embodiment, the processing subsystem (50) may further predict the type of the at least one of exercise which may be performed by the at least one user on the mat. In such embodiment, the processing subsystem (50) may predict the type of the at least one type of exercise based on the pre-defined set of exercise posture signals. In such embodiment, the pre-defined set of exercise posture signals may include the type of exercise which may be defined by the at least one user expert.

In one embodiment, the exercise posture of the at least one user may be monitored in real-time. In one specific embodiment, the system (10) may be coupled with a virtual reality (VR) device or with an augmented reality (AR) device. In such embodiment, the at least one user may be guided visually to perform the at least one exercise on the mat (20) and hence the exercise posture of the at least one user may be monitored in real time.

In another specific embodiment, the system (10) may be integrated with a voice enabled device which may be configured to assist the at least one user with a speech, which may help the at least one user to correct the posture and the angle of the at least one user in real-time. Further, such system (10) may be implemented on a visually challenged user.

In one embodiment, the system (10) may further include a transmitting module which may be operatively coupled to the processing subsystem, wherein the transmitting module may be configured to transmit a monitored exercise posture of the at least one exercise performed by the at least one user on the mat may be transmitted to at least one hand held device. In one embodiment, the hand held device may be a mobile phone, a laptop or a tablet. In such embodiment, the at least one user may keep a track on whether the at least on exercise performed by the at least one user has been performed in a right way or not based on the combined signal and the pre-defined set of exercise posture signals.

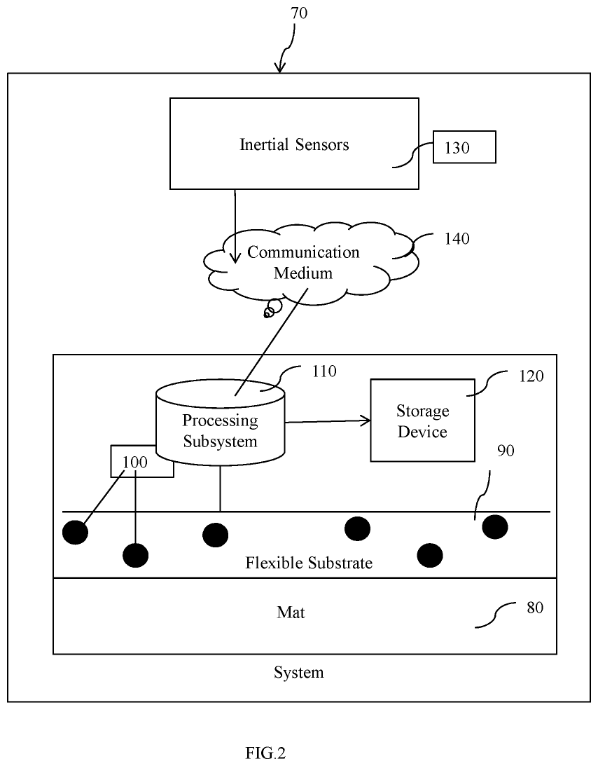

FIG. 2 is an exemplary embodiment representing a system to monitor an exercise posture of at least one user of FIG. 1 in accordance with an embodiment of the present disclosure. The system (70) includes a mat (80), wherein the mat (80) is substantially similar to a mat (20) of FIG. 1. The mat (80) may be configured to allow at least one user to perform at least one exercise on the mat (80). In one embodiment, the mat (80) may be a Pilate mat, a yoga mat, a fitness mat or a wellness mat.

Further, the system (70) includes a flexible substrate (90) which may be placed on top of the mat (80), wherein the flexible substrate (90) may include a plurality of contact sensors (100). Further, the flexible substrate (90) is substantially similar to a flexible substrate (30) of FIG. 1 and the plurality of contact sensors (100) is substantially similar to one or more contact sensors (40) of FIG. 1. Further, the at least one user may perform at least one exercise on the mat (80).

Further, while performing the at least one exerciser, the at least one user may make a contact of at least one part of a body of the at least one user with the mat (80). Further, the contact of the at least one part of the body of the at least one user may be sensed by the plurality of sensors (100). Further, the plurality of sensors (100) may generate a contact signal on sensing the contact of the at least one part of the body of the at least one user.

Further, a contact signal which may be generated by the plurality of sensors (100) may be transmitted to a processing subsystem (110), wherein the processing subsystem (110) may be operatively coupled to the flexible substrate (90). Further, the processing subsystem (110) may process the contact signal and may transmit a processed contact signal to a storage device (120), wherein the storage device (120) may be operatively coupled to the processing subsystem (110). Further, the processing subsystem (110) is substantially similar to a processing subsystem (50) of FIG. 1, and the storage device (120) is substantially similar to a storage device (60) of FIG. 1.

Further, the system (70) may include a plurality of inertial sensors (130) which may be communicatively (140) coupled to the processing subsystem (110). Further, the plurality of inertial sensors (130) may be configured to measure an angle and a posture of the at least one part of the body of the at least one user while performing at least one exercise on the mat (80). Further, depending on the angle and the posture of the at least one part of the at least one user, the plurality of inertial sensors (130) may sense the angle and the posture of the at least one part of the at least one user and may generate an angle signal. Further, a generated angle signal may be transmitted to the processing subsystem (110) for further processing of the angle sensor.

Further, the processing subsystem (110) may store the processed angle signal in the storage device (120). Further, the processing subsystem (110) may combine a stored contact signal and a stored angle signal to produce a combined signal.

Also, the storage device (120) may be stored with a pre-defined exercise posture signals which may be used by the processing subsystem (110) as a reference signal. In one embodiment, at least one user expert may perform the at least one exercise and the plurality of contact sensors (100) and the plurality of inertial sensors (130) may sense and generate a contact signal and an angle signal for the at least one exercise which may be performed by the at least one user expert. In such embodiment, the contact signal and the angle signal may be combined to generate a reference signal, wherein the reference signal may be stored as a pre-defined exercise posture signals in the storage device (120) of the system (70). Further, the processing subsystem (110) may compare the combined signal with the pre-defined exercise posture signals to determine an accurate posture of the at least one exercise which may be performed by the at least one user on the mat (80).

Further, based on a result after comparing the combined signal and with the pre-defined exercise posture signals, the at least one exercise performed by the at least one user may be determined whether the performed at least one exercise is rightly performed by the at least one user by rightly positioning the at least one part of the body of the at least one user which may be required to perform the at least one exercise. Further, the combined signal may be transmitted to at least one hand held device which may be accessed by the at least one user or by the at least one user expert. In such embodiment, the at least one user or the at least one user expert may be aware or may track whether the at least one exercise performed by the at least one user has been performed in a right way or not. Further, the at least one user expert may track and may also guide the at least one user regarding the at least one exercise which may be performed by the at least one user on the mat (80).

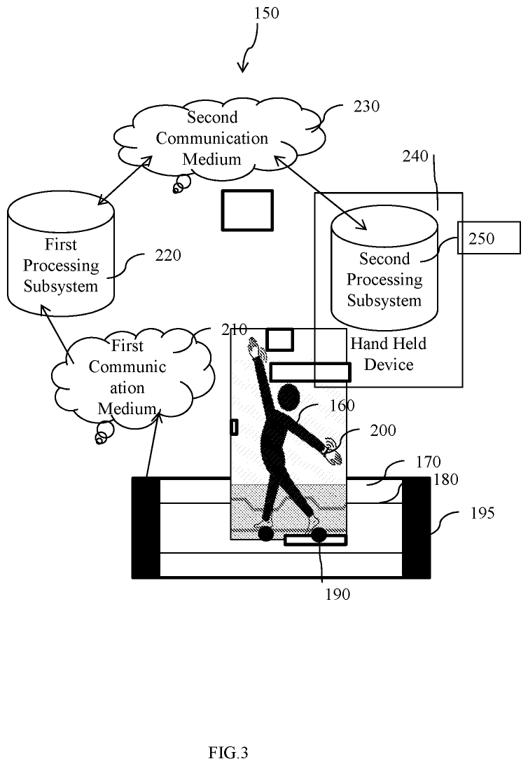

FIG. 3 is another exemplary embodiment representing a system to monitor an exercise posture of at least one user of FIG. 1 in accordance with an embodiment of the present disclosure. A user (160) may perform a yoga exercise on a mat (170), wherein the mat (170) is substantially similar to a mat (20) of FIG. 1. Further, the system (150) may include a plurality of contact sensors (190) which may be fabricated onto a flexible substrate (170), wherein the flexible substrate (170) may be placed on top of the mat (170). Further the plurality of contact sensors (190) is substantially similar to one or more contact sensors (40) and the flexible substrate (170) is substantially similar to a flexible substrate (30) of FIG. 1.

Further, the flexible substrate (170) may include a first layer which may be fabricated with a conducting path (180), wherein the conducting path may be configured to withstand an electric power. Further, the flexible substrate (170) may include a second layer which may be fabricated with the plurality of contact sensors (190) in a form of a matrix. Also, the flexible substrate (170) may include a third layer which may be place in-between the first layer and the second layer, wherein the third layer may include a plurality of slots to make a connection between the plurality of contact sensors (190) and the conducting path (180) to complete a circuit of the plurality of sensors (190).

Further, the flexible substrate (170) may include a supporting structure (195) which may be configured to receive the contact signal from the plurality of contact sensors (190). The supporting structure (195) may include a processing device, a power supply and a wireless communication device.

Further, as the user (160) may perform a yoga exercise, the user (160) may place at least on leg and at least one hand on the mat (170) to perform the yoga exercise. Further, as the user (160) may place the at least on leg and the at least one hand on the mat (170), the plurality of contact sensors (190) which may be fabricated at that particular place where the user (160) may place the at least on leg and the at least one hand, may sense a contact made by the at least on leg and the at least one hand of the user (160) with the mat (160). Further a sensed contact may be used to generate a contact signal, wherein the contact signal may describe a type of contact made by the at least on leg and the at least one hand of the user (160).

Further, the contact signal may be transmitted to a processing device which may be operatively coupled to the flexible substrate (190). Further, the processing device may process the contact signal.

Further, a processed contact signal may be transmitted to a first processing subsystem (220) through a wireless transmission device through a first communication medium (210), wherein the first processing subsystem (220) is substantially similar to a processing subsystem (50) of FIG. 1. In one embodiment, the wireless transmission device may be a Bluetooth device, a Bluetooth low energy (BLE) device or a wireless fidelity (Wi-Fi) device. Further, the first processing subsystem (220) may be communicatively coupled to the mat (170).

Further, the user (160) may be coupled with a wearable device (200), wherein the wearable device (200) may include a plurality of inertial sensors which may be communicatively coupled to the first processing device (220) through the first communication medium (210).

Further as the user (160) may perform the yoga exercise using at least one hand and at least one leg, the wearable device (200) on the user's hand may sense an angle or a position of at least one limb of the at least one hand or the at least one leg of the user (160). Further, a sensed angle of the at least one limb of the at least one hand or the at least one leg of the user (160) may be transmitted to the first processing subsystem (220) through a first communication medium (210).

Further, a yoga expert may perform at least one yoga exercise on the mat (170) prior the user (160) performs the yoga exercise. Further, the one or more contact sensors (190) may sense an expert contact signal which may be stored in the storage device of the flexible substrate (170). Further, the yoga expert may be coupled with the wearable device (200) which may include the plurality of inertial sensors which may be configured to sense and generate an expert angle signal. Further, the expert contact signal and the expert angle signal may be transmitted to the first processing subsystem (220) through the first communication medium (210) for further processing. Also, the expert contact signal and the expert angle signal may be combined to generate an expert posture signal which may be stored in the pre-defined storage device as the pre-defined set of exercise posture signals.

Further, the first processing subsystem (220) may combine the contact signal and the angle signal which may be generated while the user (160) may be performing the yoga exercise on the mat to generate a combined signal. Further, the combined signal may be compared with the pre-defined set of exercise posture signals. The combined signals may be compared with the pre-defined set of exercise posture signals to determine if the yoga exercise performed by the user (160) is done accurately or not. In one embodiment, the at least one yoga exercise done by the yoga expert may be referred to as an accurate way for doing the at least one yoga exercise based on which the user (160) performing the yoga exercise may be monitored.

Further, the combined signal may be transmitted to a hand held device (240) of the user or the yoga expert through a second communication medium (230). In one embodiment, the second communication medium (230) may be a wireless communication medium. In such embodiment, the wireless communication medium may be a Bluetooth medium, a BLE medium or a Wi-Fi medium. In another embodiment, the hand held device (240) may be the mobile phone, the laptop or the tablet. Further, based on the combined signal received on the hand held device (240), a second processing subsystem (250) may process the received combined signal based on which the yoga expert may monitor and also guide the user (160) a right way of performing the yoga exercise.

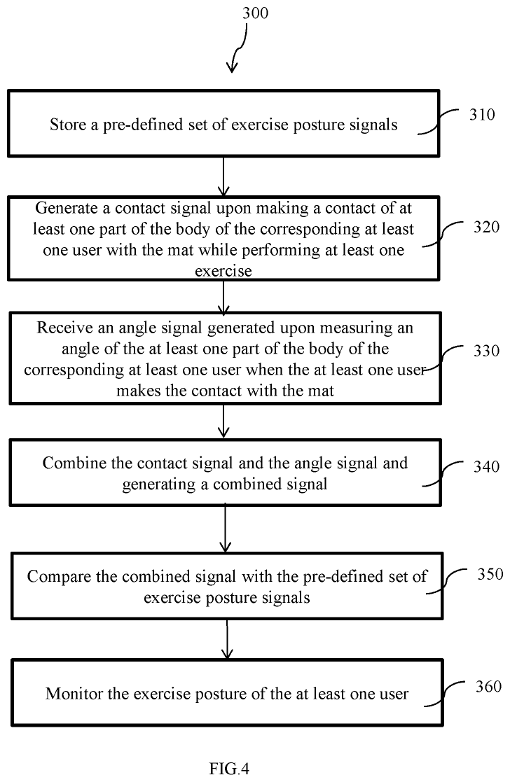

FIG. 4 is a process flow for monitoring an exercise posture of the at least one user in accordance with an embodiment of the present disclosure. The method (300) includes storing a pre-defined set of exercise posture signals (310). In one embodiment, the pre-defined set of exercise posture signals may be generated and may be stored when at least one user expert may perform at least one exercise on the mat. In another embodiment, the pre-defined set of exercise posture signals may be stored on a remote server such as a cloud server.

The method (300) also includes generating a contact signal upon making a contact of at least one part of the body of the corresponding at least one user with the mat while performing the at least one exercise (320). In one embodiment, generating the contact signal may include generating the contact signal by one or more contact signals when the user may make a contact of the at least one part of the body with the mat while performing the at least one exercise on the mat. In such embodiment, the contact signal may be generated to monitor a type of contact made by the user while performing the at least one exercise.

The method (300) further includes receiving an angle signal generated upon measuring the angle of the at least one part of the body of the corresponding at least one user when the at least one user makes the contact with the mat (330). In one embodiment, the angle signal may be generated by at least one inertial sensor. In such embodiment, the at least one inertial sensor may be operatively coupled to a wearable device. In one embodiment, the wearable device may be worn by the user on at least one part of the body while performing the at least one exercise. In another embodiment, the angle sensor may be generated to calculate an angle or a posture of the at least one user while performing the at least one exercise making use of at least one part of the body of the user.

The method (300) further includes combining the contact signal and the angle signal and generating a combined signal (340). In one embodiment, the contact angle may be combined with the angle signal to know the exact position of the user while performing the at least one exercise on the mat. In another embodiment, combining the contact signal and the angle signal and generating a combined signal may be done by using a kalman filter method or a complementary filter method.

The method (300) further includes comparing the combined signal with the pre-defined set of posture signals (350). In one embodiment, comparing the combined signal with the pre-defined set of exercise posture signals, wherein the pre-defined set of exercise posture signals may be stored in a storage device or on a remote server. In such embodiment, the remote server may be a cloud server. In one embodiment, a result may be generated based on a comparison of the combined signal with the pre-defined set of exercise posture signals.

The method (300) further includes monitoring the exercise posture of the at least one user (360). In one embodiment, the result which may be generated by comparing the combined signal and the pre-defined set of exercise posture signals may be utilised to keep a track on the at least one exercise performed by the at least one user. In such embodiment, the at least one exercise performed by the at least one user on the mat may be monitored in real time. In another embodiment, the method may further include transmitting a monitored exercise posture of the at least one exercise performed by the at least one user to a hand held device. In such embodiment, the hand device may be a laptop, a tablet or a mobile phone. In another embodiment, a user expert may user the hand held device to monitor the at least one exercise performed by the at least one user and the user expert may also provide suggestions to the at least one user regarding the at least one exercise.

Various embodiments of the present disclosure enable the system to monitor and guide the user performing an exercise in real time by the user expert which helps the user to perform the exercise in a more precise way.

Further, the type of contact made by the at least one user may be monitored very accurately with the help of one or more contact sensors. Further, the system also detects an accurate position and angle of the user body while performing the exercise which may help the user exert to monitor and train the user to improvise on performing the exercise.

Various embodiments of the present disclosure also enable the system to avoid different types of leakage current as the system uses three layers in the flexible substrate. Further, the system uses low power to enable the system for monitoring the exercise posture of the user.

Further, the system makes use machine learning models to compare the exercise posture of the user with the pre-defined exercise posture of the user expert, hence resulting in accurate detection of the exercise posture of the user.

Further, the system is flexible and can also be used on an already existing mat. Also, the system is safe for both indoor and outdoor usage. Further, the system has enhanced safety and hence user-friendly. Further, the system can be implemented on an existing mat, which makes the system cost effective.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

We claim:

1. A system (10) to monitor an exercise posture of at least one user comprising:
a mat (20) configured to allow the at least one user to perform at least one exercise;
a flexible substrate (30) placed on top of the mat (20), wherein the flexible substrate (20) comprises one or more contact sensors (40) connected to each other in a form of a matrix, and the one or more contact sensors (40) are configured to generate a contact signal upon making a contact of at least one part of a body of the corresponding at least one user with the mat (20) while performing the at least one exercise on the mat (20);
a processing subsystem (50) operatively coupled to the flexible substrate (30), and configured to:
receive the contact signal generated by the one or more contact sensors (40);
receive an angle signal generated by at least one inertial sensor upon measuring an angle of the at least one part of the body of the corresponding at least one user when the at least one user makes the contact with the mat (20) while performing the at least one exercise on the mat (20);
a storage device (60) operatively coupled to the flexible substrate (30) and the processing subsystem (50), and configured to:
store a pre-defined set of exercise posture signals;
receive and store the contact signal generated upon making a contact of the at least one part of the body of the corresponding at least one user with the mat (20);
receive and store the angle signal generated upon measuring the angle of the at least one part of the body of the corresponding at least one user when the at least one user makes the contact with the mat (20),
wherein, the processing subsystem (50) combines the contact signal and the angle signal to generate a combined signal, compares the combined signal with the pre-defined set of exercise posture signals and monitors the exercise posture of the at least one user.

2. The system (10) as claimed in claim 1, wherein the mat (20) comprises a Pilate mat, a yoga mat, a fitness mat or a wellness mat.

3. The system (10) as claimed in claim 1, wherein the flexible substrate (30) comprises:
a first layer fabricated with at least one conducting path, and configured to withstand an electric power within the conducting path;
a second layer fabricated with the one or more contact sensors (40), wherein the one or more contact sensors (40) are connected to each other in a form of a matrix; and
a third layer fabricated in-between the first layer and the second layer, and configured to insulate the conducting path of the first layer from a matrix connection of the second layer.

4. The system (10) as claimed in claim 1, wherein the flexible substrate (30) comprises an expanded polyethylene (EPE) sheet.

5. The system (10) as claimed in claim 1, wherein the processing subsystem (50) comprises a kalman filter method or a complementary filter method to combine the contact signal with the angle signal.

6. The system (10) as claimed in claim 1, wherein the at least one inertial sensor comprises at least one accelerometer sensor, at least one gyroscopic sensor or at least one magnetic sensor.

7. The system (10) as claimed in claim 1, further comprises a transmitting module operatively coupled to the processing subsystem (50), and configured to transmit the position signal to at least one hand held device.

8. A method (300) for monitoring an exercise posture of at least one user comprising:
storing a pre-defined set of exercise posture signals (310);
generating a contact signal upon making a contact of at least one part of the body of the corresponding at least one user with the mat while performing the at least one exercise (320);
receiving an angle signal generated upon measuring the angle of the at least one part of the body of the corresponding at least one user when the at least one user makes the contact with the mat (330);
combining the contact signal and the angle signal and generating a combined signal (340);
comparing the combined signal with the pre-defined set of exercise posture signals (350); and
monitoring the exercise posture of the at least one user (360).

9. The method (300) as claimed in claim 8, further comprises transmitting a monitored exercise posture of the at least one user to at least one hand held device.
